(19) United States
(12) Patent Application Publication
(10) Pub. No.: US 2007/0173887 A1
(43) Pub. Date: Jul. 26, 2007

(54) Suture and method using a suture

(76) Inventor: Gordon H. Sasaki, Pasadena, CA (US)

Correspondence Address: CHRISTIE, PARKER & HALE, LLP, PO BOX 7068, PASADENA, CA, 91109-7068, US

(21) Appl. No.: 11/657,937

(22) Filed: Jan. 24, 2007

Related U.S. Application Data
(60) Provisional application No. 60/762,104, filed on Jan. 24, 2006.

(51) Int. Cl. A61B 17/04

(52) U.S. Cl. 606/232

(57) ABSTRACT

A suspension system for rejuvenation of a human midface, the suspension system including a first suture material having a first end attached to a first needle, a second end attached to a second needle, and a first primary anchor graft having a plurality of openings through which the first suture material is inserted. The first primary anchor graft may be positioned to be about equidistant between the first end and the second end of the first suture material. The suspension system is adapted to be locatable within the human midface so as to elevate at least one malar fat pad and the first suture material may include a plurality of resistance members located on either side of the first anchor graft. The suspension system may further include a secondary braided suture attached to the first and second needles, and a guide suture inserted through the first primary anchor graft.

### CROSS-REFERENCE TO RELATED APPLICATION

This application claims priority to and the benefit of U.S. Provisional Application No. 60/762,104, filed Jan. 24, 2006, in the United States Patent Office, the entire disclosure of which is incorporated herein by reference.

### BACKGROUND OF THE INVENTION

As advances are developed in medical technology for treating almost every disorder and ailment affecting humans, it is now common for people to live to 85 to 90 years old, and it is not unusual for people to reach 100 years old. Aging has a variety of effects on the human body, especially the skin and tissue, such as natural thinning, spotting by the sun, and sagging due to the pull of gravity. As people live longer, their bodies tend to show their advanced age, often to the dismay of someone who desires to maintain a youthful, or at least a more youthful appearance, because such an appearance is generally considered to be more physically attractive. As a result of the desire to look younger, there has been increasing interest in developing procedures which treat signs of aging, the most common procedures being generally classified as plastic surgery or cosmetic surgery.

One use of plastic surgery or cosmetic surgery is directed toward treating ptosis, or the droopiness or sagging of a body part due to the long-term effect of gravity. Reducing ptosis gives the skin a healthy taut look and feel that provides a person with a more youthful physical appearance which, among other things, may also improve self esteem.

A specific area commonly addressed by plastic or cosmetic surgery is the face. Specifically, repositioning of a ptotic malar fat pad represents a key procedure for midface rejuvenation. The malar fat pad is the tissue essentially comprising the cheek and extending in a fan shape generally from below the eye socket medially to the nose and laterally along the nasolabial line. As a person ages, gravity pulls the malar fat pad downward relative to the underlying skeleton, resulting in, among other things, the cheek sagging and the creation of an exaggerated nasolabial fold. Such effects reveal aging and are often considered to make a person less physically attractive, particularly women.

As a result of these effects, a number of techniques have been developed to combat the effect of gravity on the cheek. One commonly performed technique is a superficial musculoaponeurotic system (SMAS) facelift in which the muscular system is manipulated during rejuvenation plastic or cosmetic surgery. However, the standard SMAS facelift is unable to effectively reposition the malar fat pad and nasolabial fold because of the diminishing presence of SMAS extensions over the nasolabial fold. Additionally, techniques that elevate the malar fat pad at the superficial or intermediate plane of the face have been found to place tension on the malar fat pad and tend to unnaturally flatten it.

Accordingly, there is a need for a procedure to treat midface ptosis which safely and effectively restores a natural youthful appearance and provides long-lasting effects. More specifically, there is a need for a procedure of repositioning a ptotic malar fat pad to reverse the gravitational effects of aging while maintaining a healthy, natural appearance of the face.

### SUMMARY OF THE INVENTION

A suspension system for rejuvenation of a human midface is provided, the suspension system including a first suture material having a first end attached to a first needle and a second end attached to a second needle and a first primary anchor graft having a plurality of openings through which the first suture material is inserted. The first primary anchor graft may be positioned to be about equidistant between the first suture first end and the first suture second end of the first suture material. The suspension system is adapted to be locatable within the human midface so as to elevate at least one malar fat pad and the first suture material may include a plurality of resistance members located on either side of the first anchor graft. The suspension system may further include a secondary suture material having a first end attached to the first needle and a second end attached to the second needle. The first suture material may be Prolene, the secondary suture material may be braided, and the first primary anchor graft may be polytetrafluoroethylene. Additionally, the primary anchor graft may be between about 6-12 mm long and may be between about 1.5-2.5 mm wide.

The suspension system may also include a secondary anchor graft having a plurality of openings adapted to receive the first suture material. The first secondary anchor graft may be located adjacent the first end and the second end of the first suture material such that the first suture material may be secured within a patient. The secondary anchor graft may be polytetrafluoroethylene and may be substantially square having sides of about 4 mm.

The suspension system may also include a guide suture inserted through the first anchor graft, the guide suture adapted to locate the suspension system within the human midface. Additionally, the plurality of resistance members may be knots, and specifically, fisherman nail knots. In one exemplary embodiment, there are five resistance members located on either side of the first anchor graft.

Also provided is a method for rejuvenation of a midface of a human patient using a suspension system, the suspension system including a first suture material having a first end attached to a first needle and a second end attached to a second needle, and a first anchor graft having a plurality of openings through which the first suture material is inserted. The first anchor graft may be positioned to be about equidistant between the first end and the second end of the first suture material. The method includes locating a first dot incision and a second dot incision on the midface, the second dot incision being spaced from the first dot incision and locating a temporal incision on a scalp of the human patient. The first needle is introduced into the first dot incision, passing the first needle subcutaneously through the midface, and exiting the first needle from the temporal incision. The second needle is then introduced into the second dot incision, passing the second needle subcutaneously through the midface in a direction substantially parallel to a path of the first needle, and exiting the second needle from the temporal incision. The first suture material is then secured to a second anchor graft such that the suspension system elevates a malar fat pad of the human patient.

### BRIEF DESCRIPTION OF THE DRAWINGS

FIG. 1 is a front view of the approximate location of a malar fat pad on a human face as well as an approximate location for a suture suspension system of the present invention.

FIG. 2 is a front view of some effects of aging on a human midface.

FIG. 3 shows an exemplary embodiment of a suture suspension system of the present invention.

FIG. 4 is a side view of the suture suspension system of FIG. 3 located within a patient.

FIG. 5 is a schematic view of an exemplary orbital rim suture of the present invention.

FIGS. 6 and 7 show implementation of a suture suspension system of the present invention using an open technique.

FIG. 8 is a schematic view of an implemented suture suspension system of the present invention post-surgery.

FIG. 9 is a schematic view of an exemplary lower anchor graft of the present invention.

FIG. 10 is a schematic view of an exemplary upper anchor graft of the present invention.

### DETAILED DESCRIPTION OF THE INVENTION

Embodiments of the present invention are directed to apparatus and methods for repositioning of the malar fat pad to achieve a rejuvenated appearance of the human face. More specifically, one or more of the embodiments of the present invention provide a volumetric elevation of a malar fat pad, thereby creating a more youthful triangular midface shape, lessening the fullness lateral to the nasolabial crease, filling the hollows of the midface, and smoothing out infraorbital areas.

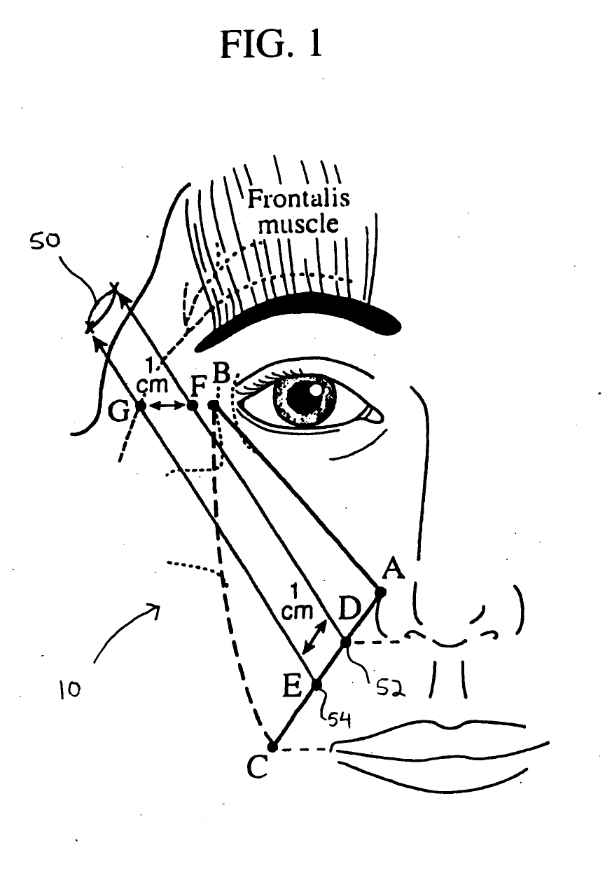

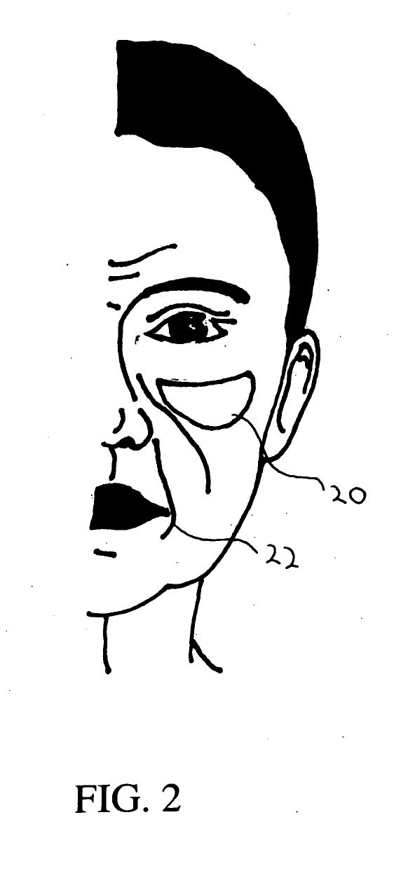

Referring to FIG. 1, a malar fat pad 10 constitutes a significant portion of the human cheek and covers a fan-shaped region generally extending from the outer lower eye (point B) medially down toward the nose (point A) and laterally along the nasolabial crease (point C). The malar fat pad 10 is a discrete, thickened triangular-shaped portion of subcutaneous fat that lies superficial and loosely attached to underlying SMAS extensions. As a person ages, the effect of gravity on the malar fat pad 10 causes, among other undesirable effects, shifts in tissue to produce a malar “bag” 20 and a more pronounced nasolabial crease 22, as shown in FIG. 2. It has been found that elevating the malar fat pad 10 significantly reduces these undesirable effects of aging and provides a person's face with a more youthful appearance.

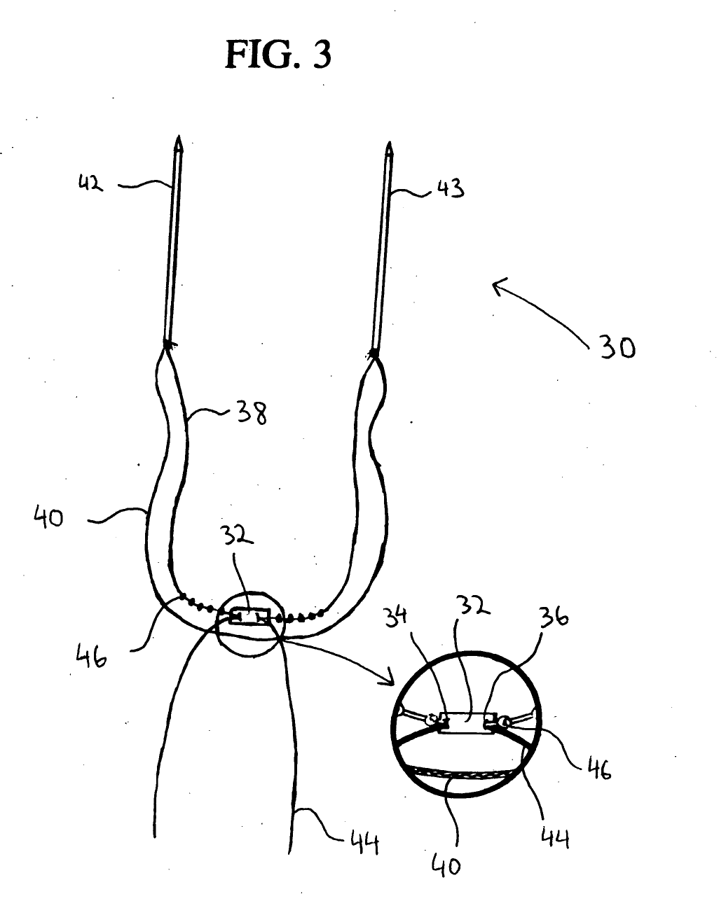

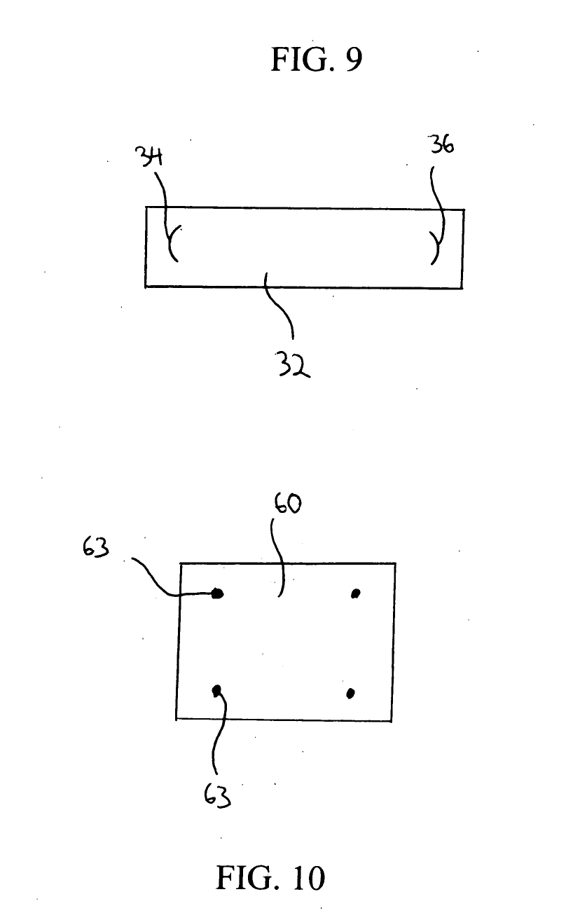

Referring to FIG. 3, an exemplary suture suspension system 30 of the present invention includes a lower anchor graft 32 (FIG. 9) which serves as a base for the suture suspension system. In one exemplary embodiment, the lower anchor graft 32 is generally rectangular and includes two perforated openings 34, 36, the openings generally located adjacent opposite longitudinal ends of the lower anchor graft. The lower anchor graft 32 may be between about 8-12 mm in length and between about 1.5-2.5 mm in width and may be made from any biocompatible material (permanent or absorbable) sufficient to elevate and support the malar far pad 10 in connection with supporting sutures as described in more detail below. In one exemplary embodiment, the lower anchor graft 32 may be expanded polytetrafluoroethylene (ePTFE), such as GORE-TEX®. Although specific dimensions and properties of the lower anchor graft 32 are provided for illustrative purposes, the dimensions and properties of the anchor graft are not limited to those provided, and one of ordinary skill in the art will appreciate that the lower anchor graft may have various dimensions and configurations which allow it to reposition the malar fat pad when the suture suspension system 30 is implemented into a patient.

With continued reference to FIG. 3, a suspension suture 38 is inserted through each opening 34, 36 in the lower anchor graft 32. The suspension suture 38 material is not critical, but may be, for example, 4-0 (about 0.15 mm diameter) synthetic, nonabsorable polypropylene, such as PROLENE®. However, an absorbable suture material such as polydioxanone may also be used. A plurality of resistance members 46 may be tied into the suture material on either side of the lower anchor graft 32 to provide additional resistant sites for tissue ingrowth and encapsulation which prevent slippage. The resistance members 46 may be between about 50%-100% larger than the diameter of the suspension suture 38 and may either be integral with the suspension suture or may be separate components. In one exemplary embodiment, the resistance members 46 are fisherman nail knots. Alternatively, the resistance members may also be points of external or internal expansion on or within the suspension suture 38. The specific number of resistance members 46 is not critical, but in one exemplary embodiment, there may be five resistance members located on each side of the lower anchor graft 32

Each end of the suspension suture 38 is attached to an appropriate suturing needle, for example, first and second 10-cm Keith triangular point needles 42, 43. The suspension suture may be made of permanent material (i.e., GORE-TEX®) or long-acting, but disolvable suture (polydioxANONE)). In addition to the suspension suture 38, each end of a braided suture 40 may also be attached to each needle 42, 43. As described in more detail below, the braided suture 40 serves as a means to eliminate irregularities in the skin after the suture suspension system 30 has been implemented in a patient. The braided suture 40 material may be an absorbable material, such as VICRYL®. The attachment of the suspension suture 38 and the braided suture 40 to the needles 42, 43 creates a double-stranded suture system.

A guide suture 44 may also be inserted through the openings 34, 36 in the lower anchor graft 32 and be generally directed in a direction opposite the suspension suture 38. As will be described in more detail below, the guide suture serves to better position the lower anchor graft 32 within a patient and, if necessary, to remove the suture suspension system 30 if the lower anchor graft is positioned in an undesired location In one exemplary embodiment, the guide suture 44 material may also be a 4-0 synthetic, nonabsorable polypropylene, and the guide suture may be a different color from the suspension suture 38 to allow the guide suture to be differentiated from the suspension suture. Although specific materials for the suspension suture 38, the braided suture 40, and the guide suture 44 have been described, the sutures are not limited thereto and any appropriate suture material, permanent or temporary, may be used.

The suture suspension system 30 may be implemented into a patient using a “closed” technique, which is minimally invasive and involves making only a few minor incisions, or using an“open” technique which involves more extensive cutting and peeling back of the skin to visibly expose the operating region.

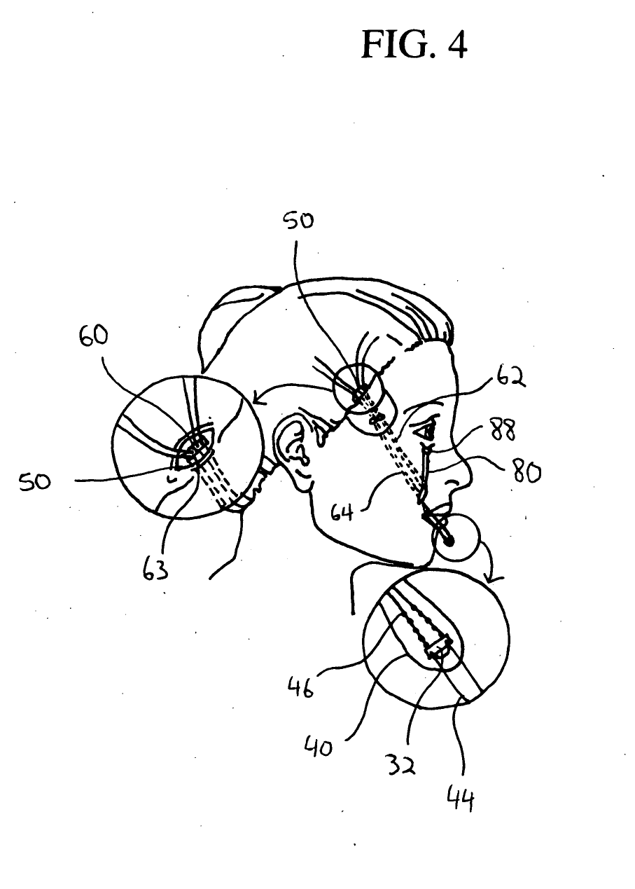

Referring to FIGS. 1 and 4, the closed technique for implementing the suture suspension system 30 includes making a temporal incision 50 behind the temporal hairline. The temporal incision 50 serves as an exit site for sutures as described in more detail below, and allows the sutures to be secured within the patient. The specific size and location of the temporal incision 50 is not critical, but in one exemplary embodiment, the incision may be about 1.5 cm in length and may be made about 1 cm behind the temporal hairline. Iris scissors may be used to create a small pocket with the tissue layers under the incision to allow easier securing of the sutures. Upper and lower dot incisions 52, 54 which serve as entry sites for creating sutures, are made along or adjacent the nasolabial crease, the incisions spaced about 1 cm apart. Again, the specific location of the upper and lower dot incisions 52, 54 is not critical, but the incisions should be located such that a direct subcutaneous path oriented generally perpendicularly to the nasolabial crease 22 from the dot incisions to the temporal incision 50 does not negatively interfere with any facial muscular or nervous structure. The dot incisions 52, 54 may be made with, for example, a No. 11 blade and, if necessary, the incisions may be widened and deepened into the subcutaneous tissue with iris scissors to allow easier entry of the needle.

The first needle 42 is percutaneously introduced into the upper dot incision 52 and passed through the pinched soft tissue, insuring uniform penetration, and oriented a few millimeters above an upper jaw bone in a superolateral direction generally perpendicular to the nasolabial crease 22 (FIG. 2). As the first needle 42 is passed progressively toward the temporal incision 50, the subcutaneous tissue is pinched along a safe pathway between the upper dot incision 52 and the temporal incision, allowing the needle to remain in the fatty subcutaneous plane before it is retrieved at the temporal incision. In one exemplary embodiment, the pathway between the upper and lower dot incisions 52, 54 and the temporal incision 50 may be drawn on the exterior surface of the patient's cheek prior to surgery as a guide. Similarly to the first needle 42, the second needle 43 is passed through the upper dot incision 52, but slightly inferiorly to the previous track, and retrieved at the temporal incision 50.

After the first and second needles 42, 43 have been guided from the upper dot incision 52 to the temporal incision 50, a generally U-shaped double-stranded suspension system of variable width is formed with the lower anchor graft 32 as the “base” of the “U.” The first and second needles 42, 43 may now be disengaged from the suspension suture 38 and the braided suture 40. Each protruding limb of the braided suture 40 may then be used to manipulate the system to substantially eliminate visible evidence of dimpling at the nasolabial crease 22, flattening of the anterior midcheek, and upward distortion of the upper lip. The lower anchor graft 32 may then be pulled through the skin by, for instance, steady, gentle traction on each limb of the suspension suture 38 to “seat” the lower anchor graft 32. The guide suture 44 may be used to establish the lower anchor graft 32 in a cupped configuration to anchor both the malar fat pad 10 and a lower fat pad adjacent the nasolabial crease 22. Specifically, the lower anchor graft 32 locks the suture tractions and provides an additional buttress to stabilize and maintain elevation of the malar fat pad 10. Within about three months, capsular formation around the suspension suture 38 and lower anchor graft 32 serves to reinforce the fixation. After the lower anchor graft 32 has been satisfactorily located, the guide suture 44 and the braided suture 40 may be removed from the operative field. If the lower anchor graft 32 is not positioned in a desired location, the suspension suture 38 and braided suture 40 may be cut from both needles 42, 43 and the apparatus withdrawn from the dot incision using the guide suture 44.

A second, substantially identical suture suspension system 30 may be passed in a similar fashion as the first suture suspension system as described above through the lower dot incision 54, generally paralleling the pathway of the first suture suspension system and exiting through the temporal incision 50. As shown in FIG. 4, a lower suspension suture 64 is slightly inferior to an upper suspension suture 62.

A spring-eye needle, for example, a half-circle French spring-eye needle, may then be attached to one strand of the suspension suture 38 extending from the temporal incision 50. The spring-eye needle is used to pass the suspension suture 38 strand through the deep temporal tissue and then through one of four openings 63 in an upper anchor graft 60 (FIG. 10). As described in more detail below, the upper anchor graft 60 serves to secure the suspension sutures 62, 64 within a patient's skin. This process is repeated three more times, once for each strand of the suspension suture 38, such that all four strands of the suspension suture protrude through the upper anchor graft 60. The upper anchor graft 60 may be made of material substantially similar to the lower anchor graft 32 and, in one exemplary embodiment, is between about 2-4 mm long and between about 2-4 mm wide.

The upper suspension suture 62 may then be used to elevate the upper dot incision 52 by a desired amount, usually between about 1 to 3 mm. In one exemplary embodiment, a sliding knot in the upper suspension suture 62 is used to elevate the upper dot incision 52. However, any other means sufficient to elevate the dot incision and reposition the malar fat pad 10 may be used. After the desired amount of elevation is reached, the ends of the upper suspension suture 62 are secured to the upper anchor graft 60 to ensure a stable elevation aided additionally by the lower anchor graft 32. In one exemplary embodiment, the upper suspension suture 62 may be secured with a plurality of square knots. Similarly, substantially similar means on the lower suspension suture 64 may be used to elevate the lower dot incision 54 by the desired amount, usually between about 1 to 3 mm. The ends of the lower suspension suture 64 may then be secured to the upper anchor graft 60, for example, by a plurality of square knots.

The upper anchor graft 60 is then buried using, for example, a permanent suture to attach the taught upper and lower suspension sutures 63, 64 to the tissue. In one exemplary embodiment, the permanent suture material is a 4-0 synthetic, nonabsorbable propylene. The temporal incision 50 is then closed in layers and the upper and lower dot incisions 52, 54 are approximated with a plurality of uninterrupted 7-0 (approximately 0.05 mm diameter) permanent sutures.

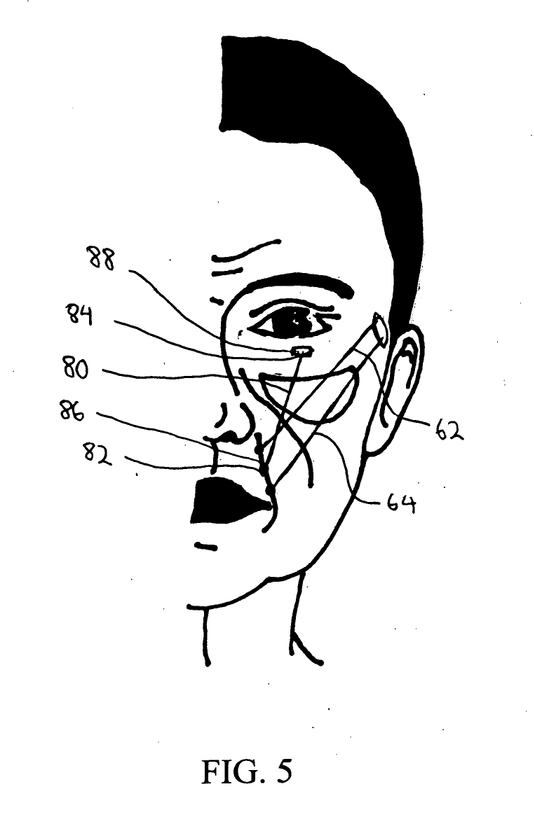

With reference to FIGS. 4 and 5, one exemplary embodiment of the present invention further includes an orbital rim suspension suture 80 which may extend subcutaneously to the orbital rim through the malar fat pad 10 from a dot incision 82 located, for example, along the nasolabial crease 22. Similarly to the previously described suspension sutures 62, 64, a suspension system is prepared including a lower anchor graft 86 having an orbital rim suspension suture 80 attached thereto and having resistance members located on either side of the lower anchor graft as described above. Ends of the orbital rim suspension suture 80 are attached to a first and second needle, respectively. In addition to the orbital rim suspension suture 80, a braided suture may be attached to the first and second needles and a guide suture may also be inserted through the lower anchor graft 86 as described above.

The orbital rim suture 80 is implemented similarly to the suspension sutures 62, 64 described above. Specifically, the first and second needles are sequentially inserted into the dot incision 82 and exited through the orbital rim incision 84 such that the lower anchor graft 86 is appropriately seated within a patient's midface, using the guide suture as necessary. Once the lower anchor graft 86 has been desirably located, a needle is used to pass the orbital rim suspension suture 80 through the tissue and through an opening of an upper anchor graft 88 similar to the previously described upper anchor graft. The orbital rim suspension suture 80 is then used to provide additional elevation to the dot incision 82, for instance, by using a sliding knot or any other appropriate means of elevation. After the desired amount of elevation is reached, the ends of the orbital rim suspension suture 80 may be secured to the upper anchor graft 88 by, for example, a plurality of square knots or by another appropriate attachment structure, and the upper anchor graft is buried within the tissue using a suture.

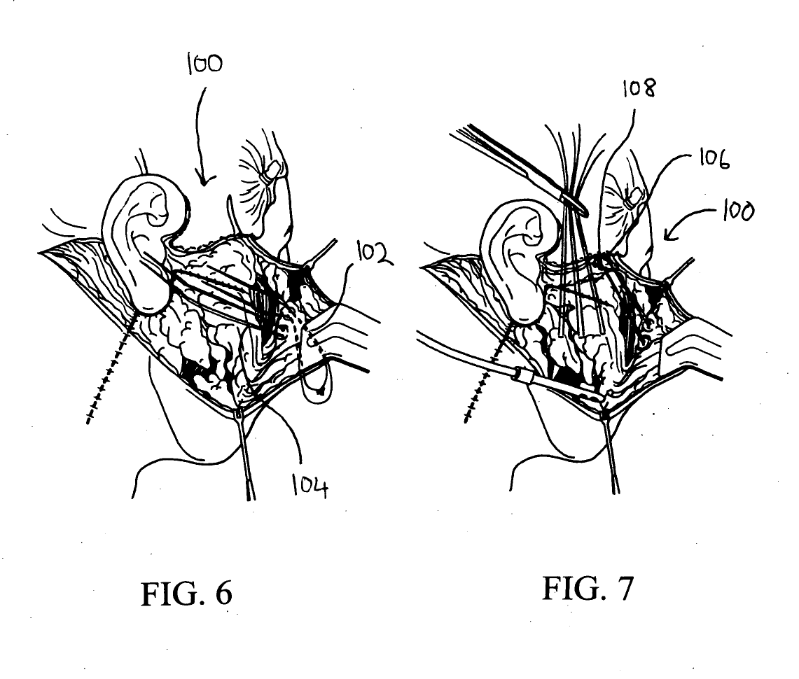

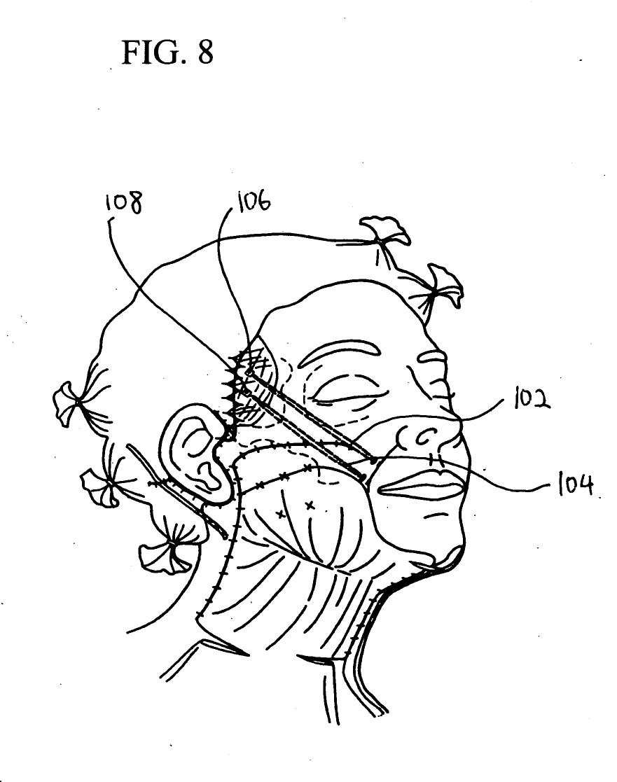

In patients with excess midface skin, descent of the malar fat pad, and laxity in the platysma muscle, an open technique may be used to implement the suture suspension system 100 as shown in FIGS. 5-8. Using the open technique, incisions are made to allow the skin to be temporarily peeled away to expose the operation site. The method to expose the operation site is within the knowledge of one of ordinary skill in the art and is not critical.

Implementing the suture suspension system 100 using the open technique is similar to implementing the system using the closed technique. However, rather than attaching the four strands of an upper and lower suspension suture 102, 104 to a single upper anchor graft, two upper anchor grafts 106, 108 may be used, one for the two strands of the upper suspension suture, and one for the lower suspension suture. Additionally, a 6.5 cm Keith triangular point needle may be used to create the sutures. An orbital rim suture 80 may also be included as described above to maximize the lift provided by the suture suspension system 100.

Although various embodiments of a suture suspension system have been described, one of ordinary skill in the art will recognize that although exemplary suture suspension systems have been described, modifications may be made to the system within the scope of the appended claims. For example, using a specific number of suspension sutures located in relatively specific places on the body, the number and location of the sutures may be varied as necessary to accommodate a patient's desires for the amount of lift and tightening of the skin and tissue. Additionally, the number and dimensions of lower and upper anchor grafts used may vary.

What is claimed is:

1. A suspension system for rejuvenation of a human midface, the suspension system comprising:
a first suture material having a first suture first end attached to a first needle and a first suture second end attached to a second needle; and
a first primary anchor graft having a plurality of openings through which the first suture material is inserted, the first primary anchor graft positioned to be about equidistant between the first suture first end and the first suture second end;
wherein the suspension system is adapted to be locatable within the human midface so as to elevate at least one malar fat pad; and
wherein the first suture material includes a plurality of resistance members located on at least one side of the first primary anchor graft.

2. The suspension system of claim 1, further comprising a secondary suture material having a secondary suture first end attached to the first needle and a secondary suture second end attached to the second needle.

3. The suspension system of claim 2, wherein the secondary suture material is braided.

4. The suspension system of claim 1, wherein the first suture material is Prolene.

5. The suspension system of claim 1, wherein the first primary anchor graft comprises polytetrafluoroethylene.

6. The suspension system of claim 1, wherein the first primary anchor graft is between about 6-12 mm long and is between about 1.5-2.5 mm wide.

7. The suspension system of claim 1, wherein the first primary anchor graft is about 10 mm long and is about 2 mm wide.

8. The suspension system of claim 1, further comprising a first secondary anchor graft, the first secondary anchor graft having a plurality of openings adapted to receive the first suture material, the first secondary anchor graft located adjacent the first suture first end and the first suture second end such that the first suture material may be secured within a patient.

9. The suspension system of claim 8, wherein the first secondary anchor graft is polytetrafluoroethylene.

10. The suspension system of claim 8, wherein the first secondary anchor graft is between about 2-4 mm long and between about 2-4 mm wide.

11. The suspension system of claim 1, further comprising a guide suture inserted through the first primary anchor graft, the guide suture adapted to aid location of the suspension system within the human midface.

12. The suspension system of claim 1, wherein the plurality of resistance members are knots.

13. The suspension system of claim 12, wherein the knots are fisherman nail knots.

14. The suspension system of claim 1, wherein there are five resistance members located on at least one side of the first anchor graft.

15. The suspension system of claim 1, wherein there are five resistance members located on both sides of the first anchor graft.

16. The suspension system of claim 1, further comprising:
a second suture material having a second suture first end attached to a third needle and a second suture second end attached to a fourth needle;
a second primary anchor graft having a plurality of openings through which the second suture material is inserted, the second primary anchor graft positioned to be about equidistant between the second suture first end and the second suture second end of the second suture material;
wherein the second suture material includes a plurality of resistance members located on at least one side of the second primary anchor graft.

17. A suspension system for rejuvenation of a human midface, the suspension system comprising:
a first suspension means having a first suspension means first end attached to a first needle and a first suspension means second end attached to a second needle;
a first anchor means having a plurality of openings through which the first suspension means is inserted, the first primary anchor means positioned to be about equidistant between the first suspension means first end and the first suspension means second end of the first suture material;
wherein the suspension system is adapted to be locatable within the human midface so as to elevate at least one malar fat pad; and
wherein the first suspension means includes a plurality of resistance members located on at least one side of the first anchor means.

18. The suspension system of claim 17, wherein the first suspension means is a suture material.

19. The suspension system of claim 17, wherein the first anchor means is an anchor graft.

20. A method for rejuvenation of a midface of a human patient using a suspension system, the suspension system including a first suture material having a first end attached to a first needle and a second end attached to a second needle, and a first anchor graft having a plurality of openings through which the first suture material is inserted, the first anchor graft positioned to be about equidistant between the first end and the second end of the first suture material, wherein the first suture material includes a plurality of resistance members located on at least one side of the first anchor graft, the method comprising:
locating a first dot incision on the midface;
locating a temporal incision on a scalp of the human patient;
introducing the first needle into the first dot incision, passing the first needle subcutaneously through the midface, and exiting the first needle from the temporal incision;
introducing the second needle into the first dot incision, passing the second needle subcutaneously through the midface in a direction substantially parallel to a path of the first needle, and exiting the second needle from the temporal incision;
securing the first suture material to a second anchor graft such that the suspension system elevates a malar fat pad of the human patient.

21. The method of claim 20, the suspension system further including a second suture material having a first end attached to a third needle and a second end attached to a fourth needle, and a third anchor graft having a plurality of openings through which the second suture material is inserted, the third anchor graft positioned to be about equidistant between the first end and the second end of the second suture material, wherein the second suture material includes a plurality of resistance members located on at least one side of the second anchor graft, the method further comprising:
locating a second dot incision on the midface, the second dot incision spaced from the first dot incision;
introducing the third needle into the second dot incision, passing the third needle subcutaneously through the midface, and exiting the third needle from the temporal incision;
introducing the fourth needle into the second dot incision, passing the fourth needle subcutaneously through the midface in a direction substantially parallel to a path of the third needle, and exiting the fourth needle from the temporal incision;
securing the second suture material to a fourth anchor graft such that the suspension system elevates a malar fat pad of the human patient.
